(19) United States
(12) Patent Application Publication
(10) Pub. No.: US 2020/0054548 A1
(43) Pub. Date: Feb. 20, 2020

(54) LIP-CARE LIPSTICK WITH ANTIBACTERIAL, ANTI-INFLAMMATORY AND ANTI-OXIDATION EFFECTS

(71) Applicant: Great You Daily Products Co.,Ltd., Guangdong (CN)

(72) Inventor: YUN XING CHAN, Guangdong (CN)

(73) Assignee: Great You Daily Products Co.,Ltd., Guangdong (CN)

(21) Appl. No.: 16/200,666

(22) Filed: Nov. 27, 2018

(30) Foreign Application Priority Data
Aug. 17, 2018 (CN) 201810942892.5

(51) Int. Cl. A61K 8/9789; A61Q 1/06; A61K 8/34; A61K 8/49; A61K 8/92; A61K 8/98; A61K 8/67; A61P 29/00; A61P 31/04; A61K 31/355; A61K 36/61

(52) U.S. Cl. CPC A61K 8/9789; A61Q 1/06; A61K 8/345; A61K 8/4946; A61K 8/922; A61K 36/61; A61K 8/678; A61P 29/00; A61P 31/04; A61K 31/355; A61K 8/987

(57) ABSTRACT

A lip-care lipstick with antibacterial, anti-inflammatory and anti-oxidation effects relates to the technical field of daily necessities. The present invention aims to provide a lip-care lipstick with antibacterial, anti-inflammatory and anti-oxidation effects. The lip-care lipstick contains a Manuka honey active ingredient ME lip-care factor and plant medicinal extracts, which can repair and prevent the cracking and peeling of lips, promote the metabolism of tender skin, inhibit the inflammation of the lips and care the lip skin. The lip-care lipstick has a significant effect on the treatment of cheilitis. The lip-care lipstick includes the following components in parts by weight: 15-30 parts of Manuka honey containing the ME lip-care factor, 0.1-1 part of dipropylene glycol, 0.5-1.5 parts of allantoin, 47-50 parts of emollient, 3-15 parts of plant essential oil, 0.15-6.5 parts of colorant, 6 parts of thickener, 5 parts of binder and 6 parts of antioxidant.

### CROSS-REFERENCE TO RELATED APPLICATION

This application claims the priority benefit of China application serial no. 201810942892.5, filed on Aug. 17, 2018. The entirety of the above-mentioned patent application is hereby incorporated by reference herein and made a part of this specification.

### BACKGROUND OF THE INVENTION

### 1. Field of the Invention

The present invention relates to the technical field of daily necessities, in particular, to a lip-care lipstick with antibacterial, anti-inflammatory and anti-oxidation effects.

### 2. Description of Related Art

The skin of the lips is different from the skin of other parts. The skin of the lips has a thin stratum corneum and has no accessory organs such as hair follicles and sebaceous glands. Many salivary glands are distributed in the stratum corneum, and its stratum corneum and stratum granulosum are thin and have less granular melanin than ordinary skin.

Cosmetic lipstick products currently on the market are mainly composed of lanolin, wax and dye. The lanolin has strong absorptivity, and can adsorb dust, bacteria, viruses and some heavy metal ions in the air onto the lip mucosa. The wax such as paraffin, white oil, or ozocerite is derived from petroleum. In addition, the lanolin and the wax have complex ingredients, easily causing allergic reactions such as cracking and peeling of the lip mucosa, and sometimes itching or slight pain in the lips. According to foreign related data, for 18.2% of young female cancer patients, causes of cancer are related to lipstick smearing. The lipstick contains a high amount of heavy metals such as lead and cadmium. Lead is accumulative, and long-term intake to the human body may lead to chronic lead poisoning, causing anemia, abdominal pain, acute kidney failure and other problems. When people drink and eat, the lipstick and these harmful substances thereon can be brought into their mouths, which directly enter the human body.

### SUMMARY OF THE INVENTION

The present invention is firstly directed to a lip-care lipstick with antibacterial, anti-inflammatory and anti-oxidation effects. The lipstick contains a Manuka honey active ingredient ME lip-care factor, which can repair and prevent the cracking and peeling of the lips, promote the metabolism of tender skin, inhibit the inflammation of the lips and care the lip skin. The lipstick has a significant effect on the treatment of cheilitis.

The present invention further provides a preparation method of the lip-care lipstick, which is simple and convenient for operation. The method can retain the Manuka honey active ingredient ME lip-care factor in the lipstick as much as possible; adding plant essential oil at the end can effectively reduce the volatilization of the essential oil; and the prepared lipstick has no granular sensation, and is smoother and more uniform in color when used.

The former technical solution provided by the present invention is as follows: a lip-care lipstick with antibacterial, anti-inflammatory and anti-oxidation effects includes the following components in parts by weight: 15-30 parts of Manuka honey containing an ME lip-care factor, 0.1-1 part of dipropylene glycol, 0.5-1.5 parts of allantoin, 47-50 parts of emollient, 3-15 parts of plant essential oil, 0.15-6.5 parts of colorant, 6 parts of thickener, 5 parts of binder and 6 parts of antioxidant.

The ME lip-care factor (Manuka Essential) refers to a composition of active ingredients such as methylglyoxal, sugar, protein, total phenol, minerals and phenolic compounds naturally present in Manuka honey, which is named the ME lip-care factor.

The emollient is one or more of wheat germ oil, prunus amygdalus dulcis oil, rosehip oil, grape seed oil, pine nut oil, limnanthes alba seed oil, diisostearyl malate, hexastearate and dipentaerythrityl tri-polyhydroxystearate.

The colorant is one or more of CI 77891, CI 19140, CI 15850, CI 45380, CI16035, CI77491, CI77492 and CI77499. The thickener is one or more of paraffin, beeswax and carnauba wax.

The binder is beeswax.

The antioxidant is vitamin E.

The plant essential oil is one or more of neroli essential oil, lemon essential oil, cinnamon essential oil, bergamot essential oil, chamomile essential oil, sage essential oil, aloe vera essential oil, jasmine essential oil, lavender essential oil and rose flower oil.

The latter technical solution provided by the present invention is as follows: a preparation method of the lip-care lipstick with antibacterial, anti-inflammatory and anti-oxidation effects as described above includes the following steps:

step 1: accurately weighing the raw materials according to a formula;

step 2: mixing and stirring the emollient and the colorant uniformly;

step 3: adding the thickener and the binder, mixing uniformly, heating to 70-90° C., and then cooling to 10-50° C.; and

step 4: adding the antioxidant, the Manuka honey containing the ME lip-care factor, the dipropylene glycol and the allantoin, mixing uniformly, grinding by a grinder, adding the plant essential oil, mixing uniformly, and pouring an obtained mixture into a mold for forming.

After the heating to 70-90° C. in step 3, the temperature is maintained constant for 15-25 minutes.

The particle size of ground particles in step 4 is ≤400 nanometers.

The present invention has the following beneficial effects:

The Manuka honey containing the ME lip-care factor is rich in vitamins A, B1, B2, B6 and E, protein and fatty acid, has the functions of nourishing and moisturizing, plays a role of soothing and resisting allergy, is vegetable oil with excellent skin care and moisturizing effects, is extremely beneficial for lip dryness, lip cracking or discomfort such as lip peeling due to climate change, and is a natural wetting agent. The Manuka honey is rich in minerals, protein and various vitamins, can promote cell growth and collagen synthesis, and is capable of effectively preventing lip allergies and eliminating swelling, dryness, inflammation and the like. Methylglyoxal naturally existing in Manuka honey is a urease inhibitor, which can effectively inhibit the activity of urease, causing bacteria to fail in producing ammonia, and causing bacteria to die in an acidic environment, thereby having an obvious antibacterial effect.

Tocopheryl acetate has a strong anti-oxidation effect, delays aging, reduces pigmentation of the lips, protects the lips from loss of moisture, effectively prevents wrinkles of the lips, and makes the lips full and bright red.

The natural plant essential oil added in the invention has a good skin care effect, is capable of decomposing accumulated pigments in the lips, accelerating the balance of nutrients and moisture and enhancing the discharge of cytotoxin, has anti-aging, moisturizing and anti-wrinkle effects, and makes the lip color looks healthier and moisturized. The plant essential oil is the only essential oil with natural care elements that can penetrate deep into the skin and the internal circulation of the human body without leaving chemical residues. It is an aromatic liquid substance that is extracted from flowers, leaves and seeds of natural plants. The molecular structure of the essential oil is very small, so it is more acceptable to the skin, and the absorption rate is almost perfect. The most important thing is that it is natural, pure and has no side effects.

### BRIEF DESCRIPTION OF THE DRAWING

FIG. 1 is a graph showing results of an antibacterial performance test.

### DESCRIPTION OF THE EMBODIMENTS

The further detailed description is made for the claims of the present invention below with reference to the specific embodiments, but does not constitute any limitation to the present invention. Any limited modifications made within the protection scope of the claims of the present invention are still within the protection scope of the appended claims.

### Embodiment 1

Step 1: Respectively weigh 15 parts of Manuka honey containing an ME lip-care factor, 0.1 part of dipropylene glycol, 1.5 parts of allantoin, 47 parts of emollient, 15 parts of plant essential oil, 0.15 part of colorant, 6 parts of thickener, 5 parts of binder and 6 parts of antioxidant.

Step 2: Mix and stir the emollient and the colorant uniformly.

Step 3: Add the thickener and the binder, mix uniformly, heat to 90° C., maintain the temperature constant for 15 minutes, and then cool to 50° C.

Step 4: Add the vitamin E, the Manuka honey containing the ME lip-care factor, the dipropylene glycol and the allantoin, mix uniformly, grind by a grinder, the particle size of ground particles being ≤400 nanometers, add the plant essential oil, mix uniformly, and pour an obtained mixture into a mold for forming.

### Embodiment 2

Step 1: Respectively weigh 30 parts of Manuka honey containing an ME lip-care factor, 1 part of dipropylene glycol, 0.5 part of allantoin, 50 parts of emollient, 3 parts of plant essential oil, 6.5 parts of colorant, 6 parts of thickener, 5 parts of binder and 6 parts of antioxidant.

Step 2: Mix and stir the emollient and the colorant uniformly.

Step 3: Add the thickener and the binder, mix uniformly, heat to 70° C., maintain the temperature constant for 25 minutes, and then cool to 10° C.

Step 4: Add the vitamin E, the Manuka honey containing the ME lip-care factor, the dipropylene glycol and the allantoin, mix uniformly, grind by a grinder, the particle size of ground particles being ≤400 nanometers, add the plant essential oil, mix uniformly, and pour an obtained mixture into a mold for forming.

### Embodiment 3

Step 1: Respectively weigh 23 parts of Manuka honey containing ME lip-care factor, 0.5 part of dipropylene glycol, 0.8 part of allantoin, 48 parts of emollient, 9 parts of plant essential oil, 4.3 parts of colorant, 6 parts of thickener, 5 parts of binder and 6 parts of antioxidant.

Step 2: Mix and stir the emollient and the colorant uniformly.

Step 3: Add the thickener and the binder, mix uniformly, heat to 80° C., maintain the temperature constant for 20 minutes, and then cool to 30° C.

Step 4: Add the vitamin E, the Manuka honey containing the ME lip-care factor, the dipropylene glycol and the allantoin, mix uniformly, grind with a grinder, the particle size of ground particles being ≤400 nanometers, add the plant essential oil, mix uniformly, and pour an obtained mixture into a mold for forming.

1 g of a lip-care lipstick prepared in Embodiment 1 was weighed, added into 9 ml of pure water, mixed and ground for 5 min, and supernatant was removed by centrifugation to prepare 10 mg/ml and 50 mg/ml test samples. The test samples passed through a microfiltration membrane to obtain sterile test sample solutions, which were placed in a refrigerator at 20° C. for later use. The sterile test sample solutions and a control group (without adding the lip-care lipstick prepared in Embodiment 1) were respectively tested for antibacterial performance, anti-oxidation performance and anti-inflammatory performance.

1. Antibacterial Performance Test

Escherichia coli was cultured by a liquid culture method to obtain a 10⁹ CFU/ml bacterial solution, the bacterial solution was diluted to 10⁴ CFU/ml, and 10 ml of the diluted bacterial solution was added to 10 μL of the 10 mg/ml or 50 mg/ml sterile test sample solution respectively. After mixing for 10 min, a mixture was coated into a culture dish, placed in a 37° C. incubator for 24 h, and taken out, and the number of colonies was calculated.

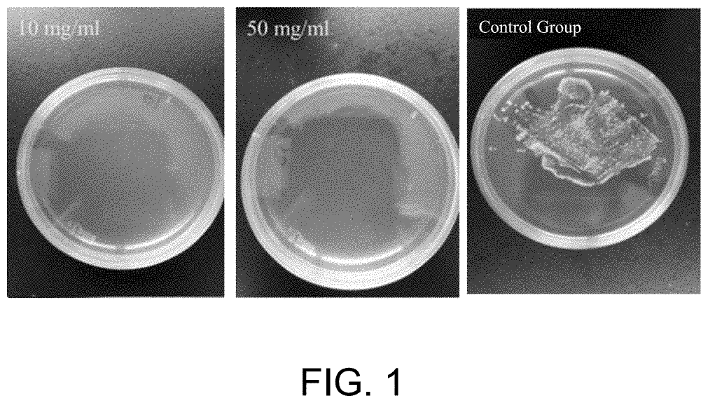

Detailed results are shown in Table 1 and FIG. 1:

TABLE 1
Results of antibacterial performance determination test

| Test Group | CFU/ml |
|---|---|
| Control group | 1083 ± 081 |
| 10 mg/ml sterile test sample solution | 0 |
| 50 mg/ml sterile test sample solution | 0 |

The test proves that the lip-care lipstick obtained in Embodiment 1 of the present invention does have an antibacterial effect.

2. Anti-Oxidation Test

H₂DCF-DA is very sensitive to hydrogen peroxide, and can be used to detect active oxides produced in cells. H₂DCF-DA per se has not fluorescence. It can be added to a culture solution and enter the cells through the cell membrane via diffusion. Intracellular enzyme esterase deacetylates the H₂DCF-DA to form polar compounds. When a peroxide is present in the cells, H₂DCF is oxidized to form a fluorescent product (DCF). As the amount of intracellular ROS increases, more H₂DCF is oxidized into the fluorescent product (DCF), and therefore, the amount of the intracellular ROS produced can be known by detecting the fluorescence intensity of DCF.

A 1×10⁵ cells/well cell culture solution was taken to 96 wells and cultured at 37° C. for 24 h; 10 μM/ml H₂DCF-DA was added, and the mixture was cultured at 37° C. for 24 h; after 1 mM H₂O₂ was added into the 10 mg/ml or 50 mg/ml sterile test sample solution, the mixture was cultured at 37° C. for 1 h in the dark; after the cells were washed with PBS, the amount of ROS was measured by a fluorescence spectrophotometer, the test was repeated for 3 times, and results were averaged. The detailed results and standard deviations are shown in Table 2:

TABLE 2
Amounts of ROS in anti-oxidation performance test

| Group | Average (nM DCF) | Standard Deviation |
|---|---|---|
| Control group | 1.31 | 0.11 |
| H₂O₂ | 1.92 | 0.41 |
| 10 mg/ml sterile test sample | 1.88 | 0.26 |
| solution | | |
| 50 mg/ml sterile test sample | 1.84 | 0.23 |
| solution | | |

The test proves that the lip-care lipstick obtained in Embodiment 1 has anti-oxidation property. The amount of ROS in the 10 mg/ml sterile test sample solution was 1.88±0.26, and the oxidation reaction was reduced by 2% as compared with the control group; and the amount of ROS in the 50 mg/ml sterile test sample solution was 1.84±0.23, and the oxidation reaction was reduced by 4%.

3. Anti-Inflammatory Performance Test

5×10⁵ cell/well BV-2 microglia cells were cultured in a 24-well plate. After culture for 24 h, the 10 mg/ml or 50 mg/ml sterile test sample solution was added, and culture was carried out for 24 h. An OD₅₉₅ absorbance was measured, the test was repeated for 3 times, and results were averaged.

The detailed results are shown in Table 3:

TABLE 3
Amounts of NO in anti-inflammatory performance test

| Group | Average (uM) | Standard Deviation |
|---|---|---|
| Control group | 4.3 | 1.1 |
| LPS | 41.2 | 6.1 |
| 10 mg/ml | 28.4 | 5.6 |
| 50 mg/ml | 20.6 | 5.1 |

The test proves that the lip-care lipstick obtained in Embodiment 1 has anti-inflammatory property. The amount of NO in the 10 mg/ml sterile test sample solution was 28.4±5.6, and the inflammation reaction was reduced by 31% as compared with the control group; and the amount of NO in the 50 mg/ml sterile test sample solution was 20.6±5.1, and the inflammation reaction was reduced by 50%.

The above are only exemplary embodiments of the present invention. Any modifications, equivalent substitutions, improvements and the like made within the spirit and scope of the present invention shall be included within the protection scope of the present invention.

What is claimed is:

1. A lip-care lipstick with antibacterial, anti-inflammatory and anti-oxidation effects, comprising the following components in parts by weight: 15-30 parts of Manuka honey containing an ME lip-care factor, 0.1-1 part of dipropylene glycol, 0.5-1.5 parts of allantoin, 47-50 parts of emollient, 3-15 parts of plant essential oil, 0.15-6.5 parts of colorant, 6 parts of thickener, 5 parts of binder and 6 parts of antioxidant.

2. The lip-care lipstick with the antibacterial, anti-inflammatory and anti-oxidation effects according to claim 1, wherein the emollient is one or more of wheat germ oil, sweet almond oil, rosehip oil, grape seed oil, pine nut oil, meadowfoam seed oil, diisostearyl malate, hexastearate and dipentaerythrityl tri-polyhydroxystearate.

3. The lip-care lipstick with the antibacterial, anti-inflammatory and anti-oxidation effects according to claim 1, wherein the colorant is one or more of CI 77891, CI 19140, CI 15850, CI 45380, CI16035, CI77491, CI77492 and CI77499.

4. The lip-care lipstick with the antibacterial, anti-inflammatory and anti-oxidation effects according to claim 1, wherein the thickener is one or more of paraffin, beeswax and carnauba wax.

5. The lip-care lipstick with the antibacterial, anti-inflammatory and anti-oxidation effects according to claim 1, wherein the binder is beeswax.

6. The lip-care lipstick with the antibacterial, anti-inflammatory and anti-oxidation effects according to claim 1, wherein the antioxidant is vitamin E.

7. The lip-care lipstick with the antibacterial, anti-inflammatory and anti-oxidation effects according to claim 1, wherein the plant essential oil is one or more of neroli essential oil, lemon essential oil, cinnamon essential oil, bergamot essential oil, chamomile essential oil, sage essential oil, aloe vera essential oil, jasmine essential oil, lavender essential oil and rose flower oil.

8. A preparation method of the lip-care lipstick with the antibacterial, anti-inflammatory and anti-oxidation effects according to claim 1, comprising the following steps:
step 1: accurately weighing raw materials according to a formula;
step 2: mixing and stirring the emollient and the colorant uniformly;
step 3: adding the thickener and the binder, mixing uniformly, heating to 70-90° C., and then cooling to 10-50° C.; and
step 4: adding the antioxidant, the Manuka honey containing the ME lip-care factor, the dipropylene glycol and the allantoin, mixing uniformly, grinding by a grinder, adding the plant essential oil, mixing uniformly, and pouring an obtained mixture into a mold for forming.

9. The preparation method of the lip-care lipstick with the antibacterial, anti-inflammatory and anti-oxidation effects according to claim 8, wherein after the heating to 70-90° C. in the step 3, the temperature is maintained constant for 15-25 minutes.

10. The preparation method of the lip-care lipstick with the antibacterial, anti-inflammatory and anti-oxidation effects according to claim 8, wherein the particle size of ground particles in step 4 is ≤400 nanometers.
